(19) United States
(12) Patent Application Publication
(10) Pub. No.: US 2008/0102056 A1
(43) Pub. Date: May 1, 2008

(54) Marketing System for Improving Patient Compliance and Methods and Devices Relating to Same

(75) Inventors: Williamson Ziegler Bradford, San Francisco, CA (US); James Pennington, Lafayette, CA (US); Stephen Rosenfield, San Francisco, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: INTERMUNE, INC., Brisbane, CA (US)

(21) Appl. No.: 10/543,466

(22) PCT Filed: Feb. 2, 2004
(86) PCT No.: PCT/US04/03083

§ 371 Date: Mar. 26, 2006

Related U.S. Application Data
(60) Provisional application No. 60/444,813, filed on Feb. 3, 2003.

(51) Int. Cl. A61K 38/21; G06Q 50/00; A61P 11/00; A61M 5/14; G06Q 30/00

(52) U.S. Cl. 424/85.5; 705/14; 604/891.1; 604/502; 604/181

(57) ABSTRACT

The present invention provides a marketing system and methods for marketing a dosage form comprising interferon gamma (IFN-γ) to a patient in need thereof, for the treatment of idiopathic pulmonary fibrosis (IPF). The present invention further provides a method for increasing patient compliance. The present invention further provides methods of treatment and dosage forms compatible for use in the marketing systems and methods of the invention.

### FIELD OF THE INVENTION

The present invention is in the field of marketing of pharmaceutical products.

### BACKGROUND OF THE INVENTION

Pulmonary fibrosis can be caused by a number of different conditions, including sarcoidosis, hypersensitivity pneumonitis, collagen vascular disease, and inhalant exposure. The diagnosis of these conditions can usually be made by careful history, physical examination, chest radiography, including a high resolution computer tomographic scan (HRCT), and transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

The logic of dividing idiopathic interstitial pneumonias into these categories is based not only on histology, but also on the different response to therapy and prognosis for these different entities. DIP is associated with smoking and the prognosis is good, with more than 70% of these patients responding to treatment with corticosteroids. NSIP patients are also frequently responsive to steroids and prognosis is good, with 50% of patients surviving to 15 years. In contrast, the UIP histologic pattern is associated with a poor response to therapy and a poor prognosis, with survival of only 3-5 years.

Idiopathic pulmonary fibrosis (IPF) is the most common form of idiopathic interstitial pneumonia and is characterized by the UIP pattern on histology. IPF has an insidious onset, but once symptoms appear, there is a relentless deterioration of pulmonary function and death within 3-5 years after diagnosis. The mean age of onset is 60-65 and males are affected approximately twice as often as females. Prevalence estimates are 13.2-20.2 per 100,000. The annual incidence is estimated to be 7.4-10.7 per 100,000 new cases per year.

Published evidence suggests that less than 20% of patients with IPF respond to steroids.

In patients who have failed treatment with steroids, cytotoxic drugs such as azathioprine or cyclophosphamide are sometimes added to the steroid treatment. However, a large number of studies have shown little or no benefit of these drugs. There are currently no drugs approved for treatment of IPF.

In addition to these problems, IPF patients must undergo therapy for the remainder of their lives.

In light of the poor prognosis of IPF, the inadequacy and toxicities of current treatment options, there exists a need in the art for improved treatment methods, and particularly treatment methods that improve patient compliance.

### Literature

WO 01/34180; Ziesche et al. (1999) N. Engl. J. Med. 341:1264-1269; du Bois (1999) N. Engl. J. Med. 341:1302-1304; U.S. Pat. No. 6,294,350; EP 795,332; King (2000) N. Engl. J. Med. 342:974-975; Ziesche and Block (2000) Wien. Klin Wochenschr. 112:785-790; Stern et al. (2001) Chest 120:213-219; Gay et al. (1998) Am. J. Respir. Crit. Care Med. 157:1063-1072; Dayton et al. (1993) Chest 103:69-73.

### SUMMARY OF TE INVENTION

The present invention provides a marketing system and methods for marketing a dosage form comprising interferon gamma (IFN-γ) to a patient in need thereof, for the treatment of idiopathic pulmonary fibrosis (IPF). The present invention further provides a method for increasing patient compliance. The present invention further provides methods of treatment and dosage forms compatible for use in the marketing systems and methods of the invention.

### BRIEF DESCRIPTION OF THE DRAWINGS

FIG. 1 depicts progression-free survival in the study patient population.

FIG. 2 depicts overall survival in the study patient population.

FIG. 3 depicts assessment of dyspnea by the transition dyspnea index over time.

### DEFINITIONS

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual, ” “host, ” “subject,” and “patient,” used interchangeably herein, refer to a mammal, particularly a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a system” includes a plurality of such systems and reference to “the dose” includes reference to one or more doses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides a marketing system for marketing a dosage form comprising interferon gamma (IFN-γ) to a patient in need thereof, for the treatment of idiopathic pulmonary fibrosis (IPF). The present invention further provides methods of marketing IFN-γ for the treatment of IPF, the method generally involving implementing the marketing system.

The present invention further provides a method of treating a patient suffering from IPF, the method generally involving administering to the patient an effective amount of IFN-γ in three bolus doses per week for a period of at least 60 weeks. The doses are preferably administered in a dosage form compatible with the marketing system and methods of the invention, where the bolus doses are delivered to the patient in a dosage form that provides for enhanced patient compliance (e.g., at least 80% patient compliance with a prescribed protocol over a period of at least about 60 weeks). Exemplary dosage forms include an implantable delivery device, preferably a programmable implanted delivery device, medication delivery pen, subcutaneous injection port delivery system, etc. Implementation of the instant methods results in enhance patient compliance, and therefore results in greater efficacy in treating IPF.

The invention solves the previously unrecognized problem that patient compliance in IFN-γ-based therapy of IPF has a significant impact upon therapeutic benefit—particularly when it results in deviation from the prescribed regimen so that the patient misses more than 20% of the doses prescribed over a 60 week period. The invention solves this problem by providing for a marketing system and methods that provide for a compliance level of a patient of at least 80% (e.g., the patient receives at least 80% of doses over a treatment period, such as at least 60 weeks).

### MARKETING SYSTEMS AND METHODS OF MARKETING

In one aspect the invention features marketing systems and methods of marketing. A marketing system generally includes marketing a delivery device for delivering IFN-γ for the treatment of IPF, to a patient suffering from IPF and/or to a physician who is prescribing IFN-γ for the treatment of IPF to a patient. A marketing system may further include the device. The invention further provides a method of increasing patient compliance, the method generally involving marketing a device to a patient suffering from IPF, wherein the device administers IFN-γ, while decreasing patient discomfort relative to subcutaneous injection using a syringe and needle. In some embodiments, the method further involves providing the drug delivery device or system. Exemplary drug delivery devices and systems include an implantable delivery device, preferably a programmable implanted delivery device, medication delivery pen, subcutaneous injection port delivery system, etc.

Exemplary marketing systems of the invention comprise one or more of the following components: 1) educational materials regarding the significance of at least 80% patient compliance (e.g., over treatment period intervals of about 60 weeks); 2) promotional materials regarding the significance of at least 80% patient compliance; 3) motivational materials (e.g., incentives; counseling; support) that increase patient compliance. Marketing material includes educational, promotional, and motivational materials including, but not limited to, printed material distributed in any forum, including printed mailings directly to affected individuals (“direct-to-consumer” or DTC mailings), advertisements in magazines, advertisements posted in waiting rooms at a doctor's office; compact disc read-only memory (CD-ROM); video cassettes; audio cassettes; digital versatile disk (DVDs); television broadcasts; radio broadcasts; electronic messages sent directly to affected individuals, e.g., via electronic mail, via an internet site, and the like; telephone messages to affected individuals; and the like.

Marketing methods of the invention generally involve marketing interferon gamma for the treatment of patients suffering from idiopathic pulmonary fibrosis, where marketing of the interferon gamma to the patients is accomplished in a manner effective to achieve at least 80% compliance with a regimen of therapy in which a therapeutically effective amount of the interferon gamma is administered to the patients three times per week for a period of at least 60 weeks.

Exemplary marketing systems of the invention include one or more of the following components: 1) information, e.g., product information, information regarding the benefits of compliance, information regarding the risk(s) of non-compliance, and patient information; 2) incentives for compliance; 3) reminders; 4) counseling/support; and 5) a device that increases patient compliance.

### Information

In some embodiments, a subject marketing system includes a message to a patient. Such message includes, e.g., one or more of: product information; information regarding the patient's current health; information regarding patient's current compliance level; information regarding risks associated with non-compliance; information regarding the benefits of compliance with a full treatment regimen; an inquiry regarding patient's emotional and physical status; general counseling and support; and an offer of additional counseling and support. Such a message is typically a DTC message. The message may be completely or partially scripted; or may be unscripted. The message can be delivered to the patient via telephone, electronic mail (email), a secure web site, and the like. The message can be delivered by a physician, a nurse, office staff, patient support staff, and call center operators. The message can be in the form of a printed material sent via mail directly to the patient. Where the message is in printed form, the message can include a questionnaire section, to be completed by the patient and returned to the sender, e.g., the physician's office. Where the message is an email message, it can also include a questionnaire, to be completed by the patient and returned via reply email to the sender.

Product information

In some embodiments, the marketing system includes product information. The product information may be various forms (e.g., a website unique resource locator (URL) directing the user to a webpage (e.g., an Internet site) which provides the instructions), where this information is typically printed on a substrate, which substrate may be one or more of: a package insert, the packaging itself, and the like, Package inserts can be in various forms, e.g., information printed on paper or other substrates, a CD-ROM, a video, a DVD, etc.

Generally, the product information includes information regarding the advantages and use of a particular drug delivery device or system. However, product information can further include information regarding IFN-γ itself.

Information regarding risks of non-compliance

In some embodiments, the marketing materials discuss and emphasize the risk(s)/dangers of non-compliance (e.g., increased risk of death, reduced life expectancy, etc.).

Information regarding the risk of non-compliance is presented in the form of a graph (e.g., life expectancy versus time), in the form of a verbal message, in the form of a written description of the risk(s), etc.

Information regarding benefits of compliance

In other embodiments, the marketing materials emphasize the advantages of a particular delivery system (e.g., a pump, a medication delivery pen, delivery via a subcutaneous injection port) in increasing patient comfort, decreasing patient discomfort, and the like. In some embodiments, the marketing materials provide information regarding use of the device, including, e.g., printed materials describing use of the device; video materials depicting a person using the device; video materials depicting a physician implementing the device; and the like.

Patient information

In some embodiments, the system further provides patient information, e.g., downloaded from patient records. Such patient information includes data on patient response during the course of treatment. Patient information that is useful to include generally relates to lung function and includes, but is not limited to, %FVC, PEF, FEV, and the like.

Patient information also includes compliance levels. Patient information regarding compliance is obtained from email messages directly from the patient, from printed materials provided to the patient and returned to the sender, e.g., the physician's office; information provided by the patient on a secure web site; patient information obtained from medical personnel; and the like. The level of compliance is calculated using standard methods.

Patient information can provide for the establishment of a patient profile, with information including, e.g., the current and past health status of the patient; the level of past patient compliance, e.g., compliance with a regimen of steroid treatments; and the like. Patient information also includes information regarding compliance during the course of IFN-γ treatment for IPF, and includes information regarding patient emotional and physical status, the number of missed doses, the timing of the missed doses, and the like. Such a patient profile is used to determine the likelihood of future compliance. Thus, in some embodiments, a subject marketing system includes a patient profile.

A patient profile is useful for tailoring a subject marketing system to individual patient's needs, and is modified as appropriate during the course of treatment. Thus, e.g., a given patient begins a course of treatment enthusiastically and adheres to a treatment regimen; however, after a period of several weeks the same patient may begin to miss occasional dosages, to become discouraged, etc. This information is used to provide a different message to the patient. For example, when a decrease in the compliance level is noted, a physician, nurse, office staff, or customer support staff may begin to call the patient one day before a dosing event is due, to provide counseling, support, information, etc.

### Incentives

In some embodiments, the marketing materials provide incentives for the use of a particular device (e.g., a pump, a medication delivery pen, delivery via a subcutaneous injection port) for the delivery of IFN-γ in the treatment of IPF. Incentives include, e.g., a free or discounted consultation if patient uses the device and registers by sending in a postcard; cash rebates; instant coupons; threshold incentives; electronic rebates; instant checks; assistance with co-pay expenses; free trial use of a drug delivery device or system; and the like. Threshold incentives include, e.g., a free re-fill at a certain point(s) during treatment. For example, the fourth re-fill is free; every tenth re-fill is free; and the like. Such free re-fills encourage a patient to continue with treatment at least up to a certain threshold, and beyond.

### Reminder System

In some embodiments, the marketing system, and method of increasing patient compliance includes a reminder system. Reminder systems include, but are not limited to, a calendaring system; a patient response form; an automatic telephone reminder system (e.g., using a pager, a voice mail, etc.); an automatic computer-based reminder system, e.g., an electronic mail system, automatic messages, voice messages, and the like; an automatic reminder system involving periodic mailings; a downloadable computer-based electronic program, e.g., for use on a personal digital assistant (PDA); and the like. In some embodiments, a reminder system provides information to the physician treating the patient, regarding patient compliance. In some embodiments, a reminder system includes one or more of a visual display, an audible alarm device, a link to an external device for sending information to a physician, a two-way informational exchange system that allows a patient to input data regarding compliance, and to receive reminders regarding compliance, and the like. Reminder systems including a audible or visual alarm provides for a signal, e.g., an audible or visual reminder to prompt patient to take medication. Exemplary reminder systems are found in, e.g., U.S. Pat. Nos. 5,157,640, 6,075,755, 6,272,532, and 5,632,242.

In some embodiments, the reminder system includes a patient response form, to be filled out by the patient upon self-administration of a dose, which form, when filled out, is sent to the patient's physician. In some patients, the need to report back to the physician after administering a dose increases compliance. In some embodiments, the patient response form is in the form of a printed material on paper, e.g., pre-addressed to the physician, which the patient fills out, then deposits in a mail box. Such printed material may be in the form of a postcard. In other embodiments, the patient response form is an electronic form sent to the physician via electronic mail, or an electronic form which is filled out on the patient's home computer on an internet site, which site is accessed by the physician or other medical personnel.

### Drug Delivery Devices and Systems

In some embodiments, the marketing system provides a device for administering IFN-γ to a patient suffering from IPF. Of particular interest are devices that decrease patient discomfort relative to the use of subcutaneous injection using a syringe and needle, particularly relative to the use of subcutaneous injection using a syringe and needle three times per week over a period of several weeks or months. Devices suitable for use in the present invention include, but are not limited to, an implantable delivery device, e.g., a programmable implanted delivery device, e.g., a pump, e.g., a programmable pump that provides for subcutaneous delivery; a medication delivery pen; and a subcutaneous injection port delivery system, which provides for administration of IFN-γ via the subcutaneous injection port, which port may be inserted into patient and left in patient for an extended period of time, thereby reducing the need for multiple injections, etc.

In some embodiments, where the system provides a device for administering IFN-γ in a manner that decreases patient discomfort and/or patient non-compliance, product information that informs the patient of the advantages of the device in reducing patient discomfort is provided. In these embodiments, product information is provided as a package insert along with the device. The product information may be various forms (e.g., a website URL directing the user to a webpage (e.g., an Internet site) which provides the instructions), where this information is typically printed on a substrate, which substrate may be one or more of: a package insert, the packaging itself, and the like. Package inserts can be in various forms, e.g., information printed on paper or other substrates, a CD-ROM, a video, a DVD, etc.

In some embodiments, the device may be provided to the patient on a trial basis. Typically the device is provided with information regarding the benefits of the device or system, use of the device or system, etc. In some of these embodiments, the device is provided to the patient along with a questionnaire, in paper or electronic form, to be filled out by the patient and returned to the sender, e.g., the physician's office. The questionnaire requests information regarding patient comfort level with the device or system; and like information. In some embodiments, patient is contacted at some point after the beginning of the use of the device or system on a trial basis to determine patient comfort with the device or system.

### Counseling/support

A subject marketing system may further include a patient counseling/support system. Such counseling or support is provided in the form of a telephone call(s) to the patient before, or during use of a device or system discussed herein; and/or visits directly to the patient.

### METHODS OF TREATING IDIOPATHIC PULMONARY FIBROSIS

The present invention provides methods of treating idiopathic pulmonary fibrosis (IPF). The methods generally involve administering an effective amount of IFN-γ to an individual having IPF.

In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met.

IFN-γ is administered in an effective amount. In some embodiments, an effective amount of IFN-γ is an amount effective to increase the probability of survival of an individual having IPF by at least about 10%, at least about 15%, at least about 20%, or at least about 25%, or more, compared to the expected probability of survival without administration of IFN-γ. Thus, the increased probability of survival of an individual having IPF and administered with an effective amount of IFN-γ is at least about 10%, at least about 15%, at least about 20%, or at least about 25%, or more, compared to the expected probability of survival without administration of IFN-γ.

In some embodiments, an effective amount of IFN-γ is an amount that reduces the risk of death in an individual with IPF. The risk of death in an individual having IPF and treated with IFN-γ is reduced at least 21-fold, at least 2.5-fold, at least 3-fold, at least 3.5-fold, or at least 4-fold, or less, compared to the expected risk of death in an individual having IPF and not treated with IFN-γ.

### INTERFERON-GAMMA

The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J. Biol. Chem. 259:6790.

IFN-γ1b (Actimmune®; human interferon) is a single-chain polypeptide of 140 amino acids. It is made recombinantly in E. coli and is unglycosylated. Rinderknecht et al. (1984) J. Biol. Chem. 259:6790-6797.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### FORMULATIONS, DOSAGE FORMS, ROUTES OF ADMINISTRATION

IFN-γ is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Effective dosages of IFN-γ can range from about 0.5 μg/m² to about 500 μm², usually from about 1.5 μg/m² to 200 μg/m², depending on the size of the patient. This activity is based on 10⁶ international units (IU) per 50 μg of protein.

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means is to measure the physiological potency of a given compound.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, dosage groups (based on administration of 200 μg IFN-γ per dose) range from about 150 μg/m² to about 80 μg/m². For example, dosage groups range from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m².

In particular embodiments of interest, IFN-γ is administered as a solution suitable for subcutaneous injection. For example, IFN-γ is in a formulation containing 40 mg mannitol/mL, 0.72 mg sodium succinate/mL, 0.10 mg polysorbate 20/mL. In particular embodiments of interest, IFN-γ is administered in single-dose forms of 200 μg/dose subcutaneously.

Multiple doses of IFN-γ can be administered, e.g., IFN-γ can be administered three times per week, four times per week, five times per week, six times per week, or daily, over a period of time ranging from about 1 year to about 2 years, or from about 2 years to about 4 years, or more. In particular embodiments of interest, IFN-γ is administered three times per week over a period of at least about 60 weeks.

In some embodiments, IFN-γ is co-administered with one or more additional agents. Suitable additional agents include corticosteroids, such as prednisone. When co-administered with IFN-γ, prednisone is administered in an amount of 7.5 mg or 15 mg daily, administered orally.

### Dosage Forms

In general, IFN-γ is provided in a dosage form that is compatible with the methods of marketing that provide for at least 80% patient compliance. Such dosage forms include, but are not limited to, an implantable drug delivery system, a medication delivery pen, and a subcutaneous injection port, which can be used together with an injection system.

### Programmable Implantable System

In one embodiment of particular interest, IFN-γ is provided in an implantable drug delivery system, preferably a system that is programmable to provide for administration of IFN-γ according to the treatment regimen of a subcutaneous infusion of IFN-γ in a bolus dose of 100 μg-300 μg three times per week (TIW), e.g., 200 μg tiw. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic). In some embodiments, the amount of IFN-γ administered between the thrice-in-week bolus doses may range from undetectable (e.g., no IFN-γ administered) to a dose that provides no therapeutic benefit (e.g., where the device may not provide for a zero flow rate of delivery, but can be adjusted to an extremely low flow rate).

In some embodiments, the invention provides a programmable implantable infusion pump, which pump is pre-programmed for delivery of a bolus dose of 200 μg IFN-γ tiw. In some of these embodiments, the pump is pre-loaded with an amount of IFN-γ formulation sufficient to deliver 200 μg IFN-γ tiw for a selected period of time, e.g., at least about 60 weeks, etc.

### Medication Delivery Pen

In some embodiments, IFN-γ is administered with a pen injector (e.g., a medication delivery pen), a number of which are known in the art. Pen injectors reduce anxiety associated with needles in some patients, and therefore decrease patient discomfort. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present invention further provides a medication delivery pen pre-loaded with a therapeutically effective amount of IFN-γ, e.g., a sufficient amount for one bolus injection of IFN-γ, in the treatment of a patient suffering from IPF according to a regimen of therapy comprising administering to the patient the therapeutically effective amount of IFN-γ as a bolus injection delivered by the pen three times per week (tiw) for at least about 60 weeks. Thus, in some embodiments, a subject medication delivery pen is pre-loaded with one or more dosage units comprising 200 μg IFN-γ per dosage unit.

### Subcutaneous Injection Port Delivery System

As a further embodiment, IFN-γ is administered via a subcutaneous injection port. To facilitate frequent or continuous subcutaneous injection of medication, subcutaneous injection ports are often used. Such injection ports extend through the skin and may remain in place for several days. Currently a major application of such injection ports is to provide chronic delivery of medication such as insulin from portable pumps. When used with a pump, a fluid line is required to connect the injection port to the portable pump. Another application of a subcutaneous injection port is to permit multiple injections without the need to re-puncture the skin. In this application, medication is injected from a standard hypodermic syringe and needle through a soft elastomer septum into the injection port which delivers the medication subcutaneously. In these embodiments, the methods comprise administering IFN-γ via a subcutaneous injection port. In some embodiments, the methods comprise installing a subcutaneous injection port in a patient; and administering IFN-γ through the injection port. Subcutaneous injection ports are known in the art. See, e.g., U.S. Pat. Nos. 3,547,119; 4,755,173; 4,531,937; 4,311,137; and 6,017,328. A combination of a subcutaneous injection port and a device for administration of IFN-γ to a patient through the port is referred to herein as “a subcutaneous injection port delivery system.” Any known subcutaneous injection port can be used in conjunction with the instant methods.

### SUBJECTS SUITABLE FOR TREATMENT

The subject methods are suitable for treatment of individuals diagnosed as having IPF. The methods are also suitable for treatment of individuals having IPF who were previously treated with corticosteroids within the previous 24 months, and who failed to respond to previous treatment with corticosteroids. Subjects that are particularly amenable to treatment with a method are those that have at least 55% of the predicted FVC. Also suitable for treatment are subject that have at least 60% of the predicted FVC, or from 55% to 70% of the predicted FVC. The percent predicted FVC values are based on normal values, which are known in the art. See, e.g., Crapo et al. (1981) Am. Rev. Respir. Dis. 123:659-664. FVC is measured using standard methods of spirometry.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Patients and Methods

### Patient Population

Between September 2000 and October 2001, 330 patients from 58 centers in the United States, Europe, Canada and South Africa were randomized into the study. The diagnosis of IPF was established according to previously described clinical, radiologic and histologic criteria.⁴ Eligible patients were those aged 20-79 years with clinical symptoms of IPF for ≧3 months, forced vital capacity (FVC) 50-90% of predicted, carbon monoxide diffusing capacity (DL_{co}) ≧25% of predicted, and room air pO₂>55 mmHg at rest (or pO₂>50 mmHg if altitude >4000 feet). The diagnosis of IPF was confirmed by high-resolution computerized tomography (HRCT) scan showing definite or probable IPF by pre-specified criteria, plus either surgical biopsy (open-lung or video-assisted thoracoscopic) showing usual interstitial pneumonia (UIP) or transbronchial biopsy to exclude other conditions. Other entry requirements included worsening of IPF within the preceding year (≧10% decrease in % predicted FVC, worsening chest X-ray, or worsening dyspnea) plus lack of improvement (<10% increase in % predicted FVC) during receipt of ≧1800 mg total of prednisone or equivalent within the 24 months prior to entry. Patients taking prednisone (≦15 mg/d) were eligible for entry into the study provided that the dose could be held constant throughout the study. All patients provided written informed consent at enrollment and the Institutional Review Board at each center approved the protocol.

Patients with any of the following were excluded: significant exposure to known fibrogenic agents, alternative etiology for interstitial lung disease, FEV₁ (forced expiratory volume in 1 second)/FVC <0.6 after bronchodilator administration, residual volume >120% predicted, active infection within 1 week preceding entry, unstable cardiovascular or neurologic disease, uncontrolled diabetes, pregnancy, lactation, or the likelihood of death within the next year. Laboratory results mandating exclusion were total bilirubin ≧1.5×upper limit normal (ULN); aspartate transaminase or alanine transaminase >3×ULN; alkaline phosphatase >3×ULN; creatinine >1.5×ULN, albumin <3.0 mg/dL, white blood cell count <2,500×10⁹/L, hematocrit <30% or >59%, and platelet count <100,000×10⁹/L. Prior treatment with any interferon was prohibited, as was use of azathioprine, colchicine, cyclophosphamide, cyclosporine, D-penicillamine, methotrexate, or N-acetyl cysteine within 6 weeks of treatment, investigational therapy for IPF within 28 days of entry, and other investigational therapies within the preceding 6 months.

### Study Design

Patients were randomized 1:1 to receive IFN-γ 1b or matching placebo, administered subcutaneously three times weekly. The randomization was stratified by cigarette smoking status and blocked by study center.

The dose of study drug was increased from 100 μg to 200 μg after 2 weeks. Bedtime administration of the study drug was recommended and pre-treatment with acetaminophen or ibuprofen required. Patient compliance was actively monitored through review of recorded injections in patient diaries and by the counting of all used medication vials. Oxygen use was also recorded in patient diaries.

Hematologic and serum chemistry tests were collected serially. After baseline measurements, arterial blood gases at rest, pulmonary function tests (FVC, FEV₁, DL_{co} corrected to hematocrit), St. George's Respiratory Questionnaire (SGRQ),¹⁹ and the Baseline Dyspnea Index/Transition Dyspnea Index (BDI/TDI)²⁰ were performed at 3-month intervals; measurement of TLC by body box plethysmography, chest x-ray, and HRCT scan were repeated at Week 48. Two expert chest radiologists who were blinded to patient identification, treatment assignment, and temporal sequence of the studies performed central and independent scoring of the extent of lung fibrosis (including honeycombing and reticular abnormalities) on the two HRCT images from each patient. A third radiologist scored in the event of discrepant interpretation.

### Statistical Issues

The primary efficacy endpoint was progression-free survival time. Progression was defined as either of the following changes from baseline, confirmed on the next visit 4-14 weeks later: ≧10% decrease in % predicted FVC or ≧5 mmHg increase in arterial-alveolar (A-a) gradient. Progression-free survival time was measured from randomization and compared between treatment groups using the likelihood ratio test from the Cox proportional hazards model, with treatment group and baseline smoking status as covariates.

Survival time was compared between treatment groups as a secondary endpoint, using the log-rank test stratified by smoking status in two pre-specified analyses: (1) all randomized patients; (2) the compliant patient cohort (i.e., patients receiving ≧80% of scheduled study drug doses). Exploratory subgroup analyses of survival were based on baseline measures of disease severity (e.g., % predicted FVC and DL_{co}) and divided the population at the median values. Multivariate analysis of covariates used Cox proportional hazards regression.

Other secondary endpoints compared the change from baseline to Week 48 in dyspnea (i.e., the TDI), DL_{co}, FVC, A-a gradient, SGRQ total score, and lung fibrosis on HRCT (better, same, worse). Two other secondary endpoints were the most severe monthly requirement for supplemental oxygen use (i.e., none, with activity, at rest) and analysis of progression-free survival using an alternate definition of disease progression (any two of the following: ≧10% decrease in % predicted FVC, ≧5 mmHg increase in A-a gradient, or ≧15% decrease in DL_{co}).

Final analysis of the data occurred at a pre-specified timepoint: 48 weeks after the 306^{th} patient was randomized. Efficacy analyses included all patients who were randomized with intention to treat. Safety analyses included all patients receiving at least one dose of study drug. Adverse events were graded according to the modified Common Toxicity Criteria of the National Institutes of Health and coded according to MedDRA preferred terms. Analysis of continuous variables used the analysis of covariance, with effects for treatment, age, sex, height, baseline value, and the inverse of baseline hemoglobin (for DL_{co}) or race (for FVC); for change in A-a gradient only treatment and baseline value were included in the model. Categorical variables were analyzed using the Cochran-Mantel-Haenszel row mean scores test, stratified by smoking status. Final (i.e., “endpoint”) evaluations were used to incorporate data from dropouts, with values carried forward from the date of last visit.

The planned sample size of 306 patients was selected to provide 94% power to detect a difference in progression-free survival time equivalent to a 20% reduction in the rate of death or disease progression at 1 year (i.e. 40% to 20%), using a two-tailed test at the 5% significance level. An independent Data Monitoring Committee regularly reviewed emerging safety and efficacy data. Patients were to continue on blinded therapy for ˜3-4 months after primary analysis of the study. Mortality is to be monitored for a total of 5 years from the date of randomization in all patients.

### RESULTS

Of the 330 patients randomized into the study, 162 received IFN-γ 1b and 168 received placebo. No imbalances in clinically relevant baseline characteristics were apparent (Table 1). The majority of patients were non-smokers (93%), Caucasian (89%), male (68%), and aged 61 to 80 years (66%). The median time since diagnosis of IPF was 312 days. Most patients were taking prednisone (76%) but did not use supplemental oxygen (58%). Baseline lung function was similar in both groups, demonstrating reduced lung volumes and abnormal gas exchange. The diagnosis of IPF was confirmed by identification of UIP on surgical lung biopsy in 62% IFN-γ 1b and 67% placebo patients, respectively. HRCT scans were interpreted as definite IPF (see Methods) in 84% vs. 83% of patients, respectively.

TABLE 1
Characteristics of the Study Population at Entry

| | IFN-γ 1b | Placebo | |
|---|---|---|---|
| Characteristic | (N = 162) | (N = 168) | P value^{a} |
| Age (years)^{b} | 63.6 ± 8.6 | 63.4 ± 8.6 | 0.8 |
| Proportion of men (%) | 71.6 | 65.5 | 0.2 |
| Ethnicity (%) | | | 0.1 |
| Caucasian | 91.4 | 86.3 | |
| Black | 1.9 | 5.4 | |
| Asian | 0 | 3.0 | |
| Hispanic | 4.9 | 4.2 | |
| Other | 1.9 | 1.2 | |
| Smoking status^{c} (%) | | | 0.2 |
| Non-smokers | 95.1 | 91.1 | |
| Smokers | 4.9 | 8.9 | |
| Days since IPF diagnosis^{b} | 425.3 ± 368.6 | 378.2 ± 295.2 | 0.2 |
| Arterial pO₂ at rest (mmHg)^{b} | 73.5 ± 10.2 | 74.1 ± 10.3 | 0.6 |
| FVC (% of predicted)^{b} | 63.9 ± 10.7 | 64.1 ± 11.3 | 0.9 |
| DL_{co} (% of predicted)^{b} | 37.2 ± 11.2 | 36.8 ± 10.6 | 0.7 |
| Use of prednisone or | 75.3 | 77.4 | 0.9 |
| equivalent (%) | | | |
| Use of supplemental | 40.7 | 31.0 | 0.1 |
| oxygen (%) | | | |

^{a}p-value is based on t-tests for continuous data and chi-square tests for categorical data
^{b}mean ± standard deviation
^{c}smokers were defined as those currently smoking or those who smoked within the year prior to study entry

The median treatment durations were 383 (range, 13-643) and 374 (range, 12-646) days in IFN-γ 1b and placebo patients, respectively. Adherence was high: an average of 93% of all scheduled doses were received, and 90% of patients complied with protocol follow-up visits through study end, even if discontinuing treatment. Sixty (33 IFN-γ 1b, 27 placebo) of the 330 (18%) randomized patients discontinued study drug treatment prematurely, due to: patient request for withdrawal: 16 vs. 16 patients, respectively; adverse event, 8 vs. 2; lung transplantation, 5 vs. 1; other reason, 1 vs. 4; investigator discretion, 3 vs. 3; use of prohibited therapy, 0 vs. 1. Seven patients (1 IFN-γ 1b, 6 placebo) who discontinued blinded study drug initiated therapy with open-label IFN-γ 1b.

### Disease Progression and Mortality

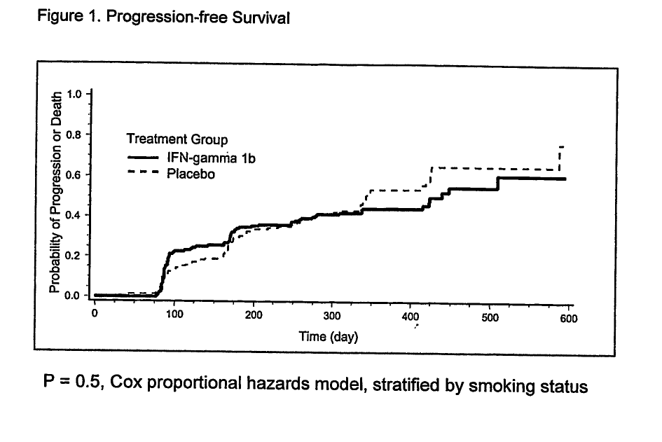

In the primary efficacy analysis, there was no significant difference in progression-free survival time in the IFN-γ 1b and placebo groups (median time to death or disease progression, 439 and 344 days, respectively; P=0.5, Cox proportional hazards model; FIG. 1). Death or disease progression occurred in 46.3% vs. 51.8% of IFN-γ 1b and placebo patients, respectively (Table 2). The majority of primary endpoint events were disease progression rather than death (88%), and the majority of disease progression events in both treatment groups (62%) were increases in A-a gradient.

TABLE 2
Progression-free Survival

| | IFN-γ 1b | Placebo | |
|---|---|---|---|
| | (N = 162) | (N = 168) | P value^{a} |
| Death or Disease Progression^{b} | 75 (46.3%) | 87 (51.8%) | 0.5 |
| Disease progression | 68 (42.0%) | 75 (44.6%) | |
| Increase in A-a gradient | 43 (26.5%) | 46 (27.4%) | |
| Decrease in FVC | 8 (4.9%) | 12 (7.1%) | |
| Both | 17 (10.5%) | 17 (10.1%) | |
| Death without disease progression | 7 (4.3%) | 12 (7.1%) | |

^{a}p value is derived from the likelihood ratio test from the Cox proportional hazards model, stratified by smoking status
^{b}the occurrence of death or disease progression was the primary endpoint of the study.
Disease progression was defined as either of the following occurrences on two consecutive occasions 4-14 weeks apart compared to baseline: ≧10% decrease in % predicted FVC or ≧5 mmHg increase in A-a gradient.

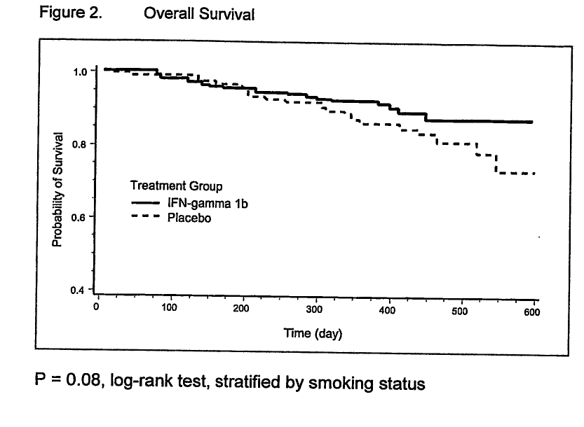

Vital status was ascertained in all enrolled patients for mortality analysis. Sixteen of 162 (9.9%) of IFN-γ 1b patients and 28 of 168 (16.7%) placebo patients died, representing a 41% relative reduction in the risk of death (P=0.08, stratified log-rank test; FIG. 2). A pre-specified analysis of the compliant patient cohort (i.e. including only those patients who received ≧80% of scheduled study drug doses) found a stronger treatment effect on survival, with a 72% reduction in the risk of death: 5 (4%) of 125 IFN-γ 1b vs. 19 (13.4%) of 142 placebo patients, respectively (P=0.01, stratified log-rank test). Exploratory subgroup analyses that dichotomized baseline lung function by median values suggested that patients with less severe lung function impairment had a greater impact of treatment on survival. In patients with baseline FVC ≧62% predicted, death occurred in 3.5% of 86 IFN-γ 1b vs. 12.5% of 88 placebo patients (P=0.04). Conversely, in patients with baseline FVC <62% (n=156), survival benefit was not apparent (death in 17.1% vs. 21.3%, respectively; P=0.6). In patients entering the study with DL_{co}>35% of predicted, mortality rates were 4.6% vs. 12.9%, respectively; P=0.06); in those with baseline DL_{co}<35%, mortality rates were 16.0% vs. 20.5%; P=0.5. In a multivariate analysis, compliance with study drug and baseline FVC were shown to be independent predictors of survival, as was study drug treatment.

The reported cause of death was related to the respiratory tract in ˜80% of patients in each treatment group. Of these, respiratory insufficiency comprised 38% and 39%, respectively, of respiratory deaths in the IFN-γ 1b and placebo groups, and progression of IPF comprised 38% and 48%, respectively. Duration of disease, gender, definite diagnosis of IPF on HRCT, mode of histopathologic diagnosis of IPF, and use of prednisone during the study period did not affect treatment group differences in survival.

No treatment effect was discernable in the mean change between baseline and Week 48 in FVC, DL_{co}, A-a gradient, change in lung fibrosis on HRCT, or using a pre-specified alternate definition of progression-free survival.

### Dyspnea and Quality of Life

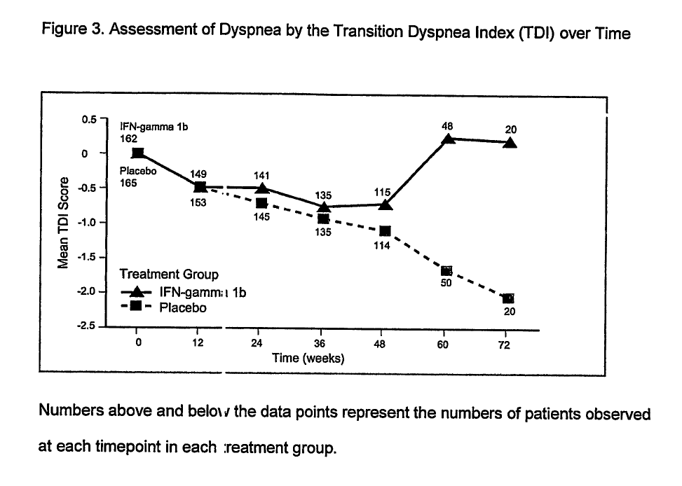

Dyspnea, as assessed by either the TDI at Week 48 or mean change from baseline to Week 48 in SGRQ total score, showed no significant treatment effect. However, divergence in TDI scores of the two treatment groups appeared to begin at Week 48 and widen thereafter, although the numbers of patients at each timepoint after Week 48 were small (FIG. 3).

Although use of supplemental oxygen was somewhat more frequent at baseline in patients receiving IFN-γ 1b (41% vs. 31%; Table 1), fewer IFN-γ 1b patients initiated new use of oxygen during the study than did placebo patients (21% vs. 35%; P=0.1).

### Safety

The incidence of treatment-emergent adverse events was high: 99% vs. 98% in IFN-γ 1b vs. placebo patients, respectively (Table 3). The most common adverse events in both groups were headache, cough and upper respiratory tract infection. Constitutional symptoms such as fever, rigor, influenza-like illness, back pain, arthralgias and myalgias were more common in IFN-γ 1b patients. Nausea and/or vomiting and dizziness were more frequent in placebo patients. Adverse events graded as severe or life-threatening events occurred in 44% vs. 34% of IFN-γ 1b and placebo patients, respectively. Those occurring in ≧5% of patients in either treatment group were: hyperglycemia (serum glucose >13.9 mmol/L; 8.6% IFN-γ 1b vs. 6.0% placebo), pneumonia (6.2% vs. 4.8%), and lymphopenia (absolute lymphocyte count <500×10⁹/L; 6.2% vs. 2.4%).

TABLE 3
Treatment-emergent Adverse Events Occurring in ≧15% of Patients

| | IFN-γ 1b | Placebo |
|---|---|---|
| | (N = 162) | (N = 168) |
| Number (%) of patients with any treatment- | 161 (99.4) | 165 (98.2) |
| emergent adverse event | | |
| Headache¹ | 86 (53.1) | 52 (31.0) |
| Upper respiratory tract infection² | 82 (50.6) | 63 (37.5) |
| Cough³ | 59 (36.4) | 59 (35.1) |
| Fever | 53 (32.7) | 16 (9.5) |
| Rigors | 53 (32.7) | 15 (8.9) |
| Fatigue⁴ | 39 (24.1) | 33 (19.6) |
| Dyspnea⁵ | 39 (24.1) | 43 (25.6) |
| Pain | 37 (22.8) | 23 (13.7) |
| Diarrhea⁶ | 37 (22.8) | 35 (20.8) |
| Arthralgia | 33 (20.4) | 23 (13.7) |
| Influenza-like illness | 31 (19.1) | 13 (7.7) |
| Myalgia | 30 (18.5) | 15 (8.9) |
| Nausea and/or vomiting⁷ | 29 (17.9) | 49 (29.2) |
| Back pain | 29 (17.9) | 20 (11.9) |
| Chest pain | 26 (16.0) | 27 (16.1) |
| NASAL CONGESTION | 25 (15.4) | 26 (15.5) |
| BRONCHITIS⁸ | 25 (15.4) | 29 (17.3) |
| DIZZINESS | 18 (11.1) | 29 (17.3) |

¹includes headache, aggravated headache, migraine, and sinus headache
²includes upper respiratory tract infection, viral upper respiratory tract infection, sinusitis, acute sinusitis, otitis media, ear infection, laryngitis, nasopharyngitis, streptococcal pharyngitis
³includes cough, aggravated cough, and productive cough
⁴includes fatigue and aggravated fatigue
⁵includes dyspnea, exacerbated dyspnea, and exertional dyspnea
⁶includes diarrhea and aggravated diarrhea
⁷includes nausea, aggravated nausea, and vomiting
⁸includes bronchitis, acute bronchitis, acute exacerbation of chronic bronchitis, and tracheobronchitis

Respiratory tract infections were frequent, occurring in 67.9% of IFN-γ 1b patients and in 56.5% of placebo patients overall. Of these, pneumonias comprised 14.8% vs. 8.3%, respectively, and unspecified respiratory tract infections, 11.7% vs. 11.3%. Respiratory tract infections that were graded by the investigator as severe or life-threatening were reported in 13 (8.0%) IFN-γ 1b and 14 (8.3%) placebo patients. Twenty-nine respiratory tract infections resulted in hospitalization in 26 (16.0%) IFN-γ 1b patients, as did 19 events in 16 (9.5%) placebo patients. Respiratory tract infections that resulted in death occurred in 3 patients in each treatment group. Only one respiratory infection, an episode of acute bronchitis/pneumonia in a patient receiving placebo, resulted in withdrawal from study drug treatment.

### REFERENCES

1. Schwartz D A, Helmers R A, Galvin J R, et al. Determinants of survival in idiopathic pulmonary fibrosis. Am J Respir Crit Care Med 1994; 149:450-4.
2. Bjoraker J A, Ryu J H, Edwin M K, et al. Prognostic significance of histopathologic subsets in idiopathic pulmonary fibrosis. American Journal of Respiratory Critical Care Medicine 1998; 157:199-203.
3. Douglas W W, Ryu J H, Schroeder D R. Idiopathic pulmonary fibrosis: Impact of oxygen and colchicine, prednisone, or no therapy on survival. Am J Respir Crit Care Med 2000;161:1172-8.
4. American Thoracic Society. Idiopathic pulmonary fibrosis: diagnosis and treatment. International consensus statement. Am J Respir Crit Care Med 2000; 161:646-664.
5. Johnson M A, Kwan S, Snell N J C, Nunn A J, Darbyshire J H, Turner-Warwick M. Randomised controlled trial comparing prednisolone alone with cyclophosphamide and low dose prednisolone in combination in cryptogenic fibrosing alveolitis. Thorax 1989; 44:280-8.
6. Raghu G, Depaso W J, Cain K, et al. Azathioprine combined with prednisone in the treatment of idiopathic pulmonary fibrosis: a prospective double-blind, randomized, placebo-controlled clinical trial. Am Rev Respir Dis 1991; 144:291-6.
7. van Oortegem K, Wallaert B, Marquette C H, et al. Determinants of response to immunosuppressive therapy in idiopathic pulmonary fibrosis. Eur Respir J 1994; 7:1950-7.
8. Selman M, Carrillo G, Salas J, et al. Colchicine, D-penicillamine, and prednisone in the treatment of idiopathic pulmonary fibrosis: a controlled clinical trial. Chest 1998; 114:507-12.
9. Flaherty K R, Toews G B, Lynch J P III, et al. Steroids in idiopathic pulmonary fibrosis: a prospective assessment of adverse reactions, response to therapy, and survival. Am J Med 2001; 110:278-82.
10. Billiau A., Vanderbroeck K. IFN-γ. In: Cytoline Reference. A compendium of cytokines and other mediators of host defense. Oppenheim J J, Feldmann M, eds. 2001, Academic Press: pp. 641-688.
11. Narayanan A S, Whithey J, Souza A, Raghu G. Effect of gamma-interferon on collagen synthesis by normal and fibrotic human lung fibroblasts. Chest 1992; 101:1326-31.
12. Clark J G, Dedon T F, Wayner E A, Carter W G. Effects of interferon-gamma on expression of cell surface receptors for collagen and deposition of newly synthesized collagen by cultured human lung fibroblasts. J Clin Invest 1989; 83:1505-11.
13. Okada T, Sugie I, Aisaka K. Effects of gamma-interferon on collagen and histamine content in bleomycin-induced lung fibrosis in rats. Lymphokine Cytokine Res 1993; 12:87-91.
14. Lack G, Gelfand E W. The role of nebulized IFN-gamma in the modulation of allergic responses. Adv Exp Med Biol 1996;409:17-23.
15. Gajewski T F, Fitch F W. Anti-proliferative effect of IFN-gamma in immune regulation. I. IFN-gamma inhibits the proliferation of Th2 but not Th1 murine helper T lymphocyte clones. J Immunol 1998;140:4245-5252.
16. Neumann B, Emmanulidis K, Stadler M, Holzmann B. Distinct function of interferon-gamma for chemokine expression in models of acute lung inflammation. Immunology 1998;95:512-521.
17. Ziesche R, Hofbauer E, Wittnann K, Petkov V, Block L-H. A preliminary study of long-term treatment with interferon gamma-1b and low-dose prednisolone in patients with idiopathic pulmonary fibrosis. N Engl J Med 1999; 341:1264-9.
18. Ziesche R, Bradford W Z, Crager M, Block L-H. Long-term survival in idiopathic pulmonary fibrosis patients treated with interferon gamma-1b (abstract). Chest 2002; 122: 75S-76S.
19. Chang J A, Curtis J R, Patrick D L, Raghu G. Assessment of health-related quality of life in patients with interstitial lung disease. Chest 1999; 116:1175-82.
20. Mahler D A, Weinberg D H, Wells C K, Feinstein A R. The measurement of dyspnea. Contents, interobserver agreement, and physiologic correlated of two new clinical indexes. Chest 1984; 85:751-758.
20a. King T E, Jr., Tooze J A, Schwarz M I, Brown K, Cherniack R M. Predicting survival in idiopathic pulmonary fibrosis. scoring system and survival model. Am J Respir Crit Care Med 2001; 164:1171-1181.
20b. Panos R J, Mortenson R L, Niccoli S A, King T E Jr. Clinical deterioration in patients with idiopathic pulmonary fibrosis: causes and assessment. Am J Med 1990; 88(4):396-404
21. Keogh B A, Bernardo J, Hunninghake G W, Line B R, Price D L, Crystal R G. Effect of intermittent high dose parenteral corticosteroids on the alveolitis of idiopathic pulmonary fibrosis. Am Rev Respir Dis 1983; 127:18-22.
22. Douglas W W, Ryu J H, Swensen S J et al. Colchicine versus prednisone in the treatment of idiopathic pulmonary fibrosis. A randomized prospective study. Am J Respir Crit Care Med 1998; 158:220-5.
23. O'Donnell K, Keogh B, Cantin A, Crystal R G. Pharmacologic suppression of the neutrophil component of the alveolitis in idiopathic pulmonary fibrosis. Am Rev Respir Dis 1987; 136:288-92.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of marketing an interferon gamma for the treatment of patients suffering from idiopathic pulmonary fibrosis, the method comprising marketing the interferon gamma to the patients in a manner effective to achieve at least 80% compliance with a regimen of therapy in which a therapeutically effective amount of the interferon gamma is administered to the patients three times per week for a period of at least 60 weeks.

2. A method of treating a patient suffering from idiopathic pulmonary fibrosis, the method comprising administering to the patient an effective amount of an interferon gamma in a dosing regimen of three bolus doses per week for a period of at least 60 weeks, wherein the bolus doses are delivered to the patient in a dosage form that provides for at least 80% compliance with the dosing regimen.

3. The method of claim 2, wherein the dosage form is selected from the group of an implantable programmable pump, a medication delivery pen, and a subcutaneous injection port delivery system.

4. A medication delivery pen loaded with an effective amount of IFN-γ for the treatment of a patient suffering from idiopathic pulmonary fibrosis (IPF) according to a regimen of therapy comprising administering to the patient the effective amount of IFN-γ as a bolus injection delivered by the pen three times per week for at least about 60 weeks.

5. An implantable programmable pump pre-loaded with an effective amount of IFN-γ for the treatment of a patient suffering from idiopathic pulmonary fibrosis (IPF) and programmed to deliver to the patient 200 μg of IFN-γ three times per week.
